(19) United States
(12) Patent Application Publication
(10) Pub. No.: US 2002/0068867 A1
(43) Pub. Date: Jun. 6, 2002

(54) System for determining the intracorporal position of a working catheter

(75) Inventors: Walter Ameling, Aachen (DE); Lothar Thieling, Erkelenz (DE); Ludwig Adams, Aachen (DE)

Correspondence Address: Stephen L. Grant, Oldham & Oldham Co., L.P.A., Twin Oaks Estate, 1225 West Market Street, Akron, OH, 44313-7188, US

(73) Assignee: BIOTRONIK MESS-UND THERAPIEGERAETE GMBH & CO

(21) Appl. No.: 09/873,767

(22) Filed: Jun. 4, 2001

(30) Foreign Application Priority Data
Jun. 7, 2000 (DE) 100 27 782.9

(51) Int. Cl. A61B 5/05

(52) U.S. Cl. 600/424; 600/374; 607/122

(57) ABSTRACT

The invention concerns a system for determining the intracorporal position of a working catheter (10) for carrying out desired working operations. The system has an intracorporal reference catheter (2) which is adapted to produce a coordinate system. The working catheter (10) has a plurality of working catheter reference units (4) which are adapted to send signals which are characteristic for the position of the working catheter (10), and the reference catheter (3) has a plurality of reference catheter reference units (14) which are adapted to receive the signals which are sent by the working catheter reference units (4). The system also has a processing unit (16) which is adapted to calculate the position and the intracorporal orientation of the working catheter (10) on the basis of signals received from the reference catheter reference units (14).

The invention concerns a system for determining the intracorporal position of a working catheter for carrying out desired working operations and an intracorporal reference catheter for producing a co-ordinate system.

### BACKGROUND OF THE ART

Catheters are used in many different ways in present day medicine. In that respect an aspect of particular interest is to be able to detect the position of the catheter, even in the implanted or inserted condition, from outside the body. That applies in particular in relation to catheters which are used for the treatment of heart conditions, in which respect also it is important to determine the position of the catheter as accurately as possible. That applies both in terms of collecting data which describe the condition of the heart so that the data can be exactly allocated; but it also applies in regard to precisely determining the position of catheters which must be moved into the correct position in preparation for the actual therapy procedures, in order to optimise the therapy.

Atrial fibrillation of the heart may be mentioned as an example of the area of use of the system referred to in the opening part of this specification in medicine. This pathological variation is characterised by an atrial rate of over 350/minute and a completely irregular heart beat sequence (absolute arrhythmia). Apart from extrasystoles and sinus tachycardia phenomena, atrial fibrillation is the most frequent rhythm disturbance in adult age with a prevalence of 0.4% in the case of adults, which rises to between two and four percent in the case of those over 60 years old. Atrial fibrillation is due to a plurality of continuously changing re-entry excitation circles in the atria of the heart. The fronts of the corresponding excitation waves circle chaotically around the shortly previously excited and therefore refractory atrium myocardium without anatomical obstacle. In that case the AV-node is confronted at a rate of between 350 and 600/minute from all directions with excitation fronts. The line delay which occurs in the AV-node finally has the result that most atrial excitations remain stuck at different depths in the AV-node and accordingly chamber activity takes place only very occasionally. That results in a reduction in the heart output by up to 30% due to the absence of pumping capacity on the part of the atrium. In addition, due to the absence of pumping capacity there is the risk of thrombosis formation in the left atrium, which in turn can result in a stroke.

This risk of a stroke, which is increased due to atrial fibrillation, may be explained at this point on the basis of some case figures. Thus, about 100,000 new stroke cases occur per annum in the Federal Republic of Germany. The most frequent cause of such strokes, in 75% of all cases, are arterial vessel occlusions, of which in turn 20% are caused by embolisms. Those embolisms mostly occur due to the above-discussed atrial fibrillation so that approximately 15% of all stroke cases, that is to say 1500 new illness cases per year, are to be attributed to atrial fibrillation. Patients with atrial fibrillation thus have an increased risk of suffering a stroke. In addition, the risk of dying with a stroke is twice as great in the case of patients with atrial fibrillation as in the case of patients without atrial fibrillation. For comparison purposes, it should also be mentioned that in the United States of America about two million people live with atrial fibrillation, of which approximately 600,000 suffer from a stroke each year, with once again about 160,000 patients dying from the stroke. In the USA the stroke is the third most frequent cause of death after cardiovascular diseases and cancer.

The known therapies for the treatment of atrial fibrillation are mostly only palliative. That means that mostly only the various consequences of atrial fibrillation are treated, without the underlying disease being eliminated. These therapies which are therefore only directed to the symptoms can be implemented for example by medication, by atrial fibrillation being treated with anti-coagulants. Those anti-coagulants reduce the risk of thrombosis formation and thus the risk of a stroke. Anti-coagulants of that kind are mostly given together with anti-arrhythmia drugs. Those drugs however can in turn increase the risk of a life-threatening ventricular tachycardia. It is also known as a form of therapy to sever the AV-node and to implant a pacemaker. Those measures serve for restoration of a normal ventricular cycle. In that respect however the cause still remains, namely atrial fibrillation, and also the risk of a stroke.

Implantation of an intracardial defibrillator is known as a further form of therapy. Such a defibrillator is capable of terminating atrial fibrillation by current shocks, so-called defibrillation. Even with this therapy however the actual cause of atrial fibrillation remains untreated. In addition the generally unforeseen current shock of defibrillation is perceived as being highly unpleasant by the patients.

The only known curative therapy hitherto is the maze procedure in which electrically insulating, linear scar tissue is produced in the endocardium in a surgical intervention on the open heart. That scar tissue prevents the occurrence of circle excitations and thus the occurrence of atrial fibrillation. The pumping function of the atrium is thus maintained and the ventricle rhythm of the heart is normalised. That operation however is very expensive and linked to a high rate of mortality and morbidity.

The systems set forth in the opening part of this specification were developed in the state of the art in order to deal with those difficulties. In that respect, the above-described therapy is implemented in a minimally invasive mode by means of ablation catheters. A system of that kind is known for example from U.S. Pat. No. 5,718,241. The subject-matter of that patent is measuring the positions of arrhythmogenic zones by means of ablation catheters and determining the position of the catheter by way of reference filters. In order to produce an electrical image on the base of refractory time and stimulation line speed measurements, the position of an ablation catheter with a tip electrode is to be determined for example by means of a reference field. As a result of that mapping procedure, a geometrical dimension (‘dimension value’) is to be determined for zones with given stimulation line properties, on the basis of which the size and position of the lesions to be produced are determined. As that known position measuring procedure in respect of a tip electrode can be implemented only successively, that is to say point by point, the geometry and the associated electrical activity of the corresponding cardiac cavity can also be determined only point by point. In addition, there is the prerequisite that there is a steady arrhythmia which is well tolerated by the patient, as, due to the principle involved, it is not possible to take measurements at the same time at various locations in the endocardium and the procedure is thus time-consuming. Producing the linear lesion is also again time-consuming as this can only be produced point by point.

Therefore the object of the present invention is to improve the usability of the system set forth in the opening part of this specification.

### SUMMARY OF THE INVENTION

In a system of the kind set forth in the opening part of this specification, that object is attained in that the working catheter has a plurality of reference units which can be detected by the sensor device.

The invention equally has a whole series of advantages. Firstly by virtue of the invention it is possible to provide for precisely determining the position and also the orientation and the configuration in space of the working catheter. The working catheter can be both a mapping catheter which measures the electrical activity of the corresponding region of the heart by means of electrodes mounted on the catheter; it may however also involve an ablation catheter which provides the corresponding regions with a lesion. In both cases however the invention permits exact positioning, orientation and establishment of the spatial configuration of the corresponding catheter not only in respect of its tip but also in respect of the entire distal portion on which are disposed reference units which can be detected by the sensor device of the reference catheter. A marked increase in positional resolution is thus possible. That in turn permits high-resolution mapping of all heart cavities in which the working catheter is disposed. It is further advantageous that precise positioning of the working catheter can be based on tried-and-tested catheters so that patient safety, positioning certainty, handling and authorisation, as are known from tried-and-tested catheters, are still retained. Accurate positioning of the ablation catheter after accurately targeted and high-resolution mapping of the endocardium which is of interest thus represents an outstanding alternative to the above-mentioned maze operation, while at the same time it is possible to achieve markedly reduced mortality and morbidity rates. Furthermore, in comparison with the maze operation, by means of the invention, in spite of the markedly higher level of accuracy of the intervention, such an intervention involves considerably lower costs.

The system according to the invention thus enables the physician to arrive at an accurate assessment of the excitation mechanisms of the endocardium to be treated and enables him to implement accurate planning of the therapy, namely accurately establishing the configurations of the lesion lines which are to be applied by means of the ablation catheter. Transfer of the therapy plan which is finally to be worked out by the physician to the patient can be considerably improved by means of the system according to the invention as the ablation catheter for applying the lesion lines can be measured just as precisely as the anatomy involved so that accurately targeted positioning of the ablation catheter and thus accurately targeted application of the lesion lines is possible.

Furthermore, by virtue of the invention it is possible to record signals, that is to say to implement the mapping operation, at the same time at various locations by means of the working catheter and its reference units, even in situations involving tachycardia which occurs for a short time or which is poorly tolerated by the patient. Thus, with just a single catheter which alternatively can also be in the form of a cage catheter, the system can provide for three-dimensional mapping of the region of the heart which is of interest, without this requiring a large amount of time which cannot be tolerated in such a tachycardia situation.

It can thus be established that the system according to the invention permits an accurate three-dimensional image of the heart cavity to be investigated and a representation related thereto of the electrical activity by the availability of a plurality of reference units on the working catheter. In addition, the system according to the invention provides an ablation catheter which, thanks to the system according to the invention, can produce linear lesions without re-positioning, as by means of the system it can be placed precisely and without additional X-ray measures, insofar as the configuration of the ablation catheter, which is detected by the reference catheter, is blended for example continuously into a three-dimensional representation of the corresponding heart cavity, which representation can be made available for example by means of a suitable mapping catheter.

A further embodiment of the invention is distinguished in that the working catheter is a catheter which can be fixedly implanted in a body and which carries electrodes of a cardiac pacemaker or a defibrillator. The advantages of this embodiment are in particular that the intracorporal reference catheter of the system makes it possible to exactly determine the intracorporal position of the catheter carrying the electrodes, without involving the patient being exposed to X-ray radiation.

A further preferred embodiment of the invention is distinguished in that the reference units are arranged on the working catheter in such a way that the position and/or the orientation of the working catheter in the co-ordinate system can be detected by the sensor device. Therefore, by means of this embodiment, it is not only possible to establish the precise position in the body, but it is also possible to detect a possible rotation of the catheter and thus a possible change in the orientation or the spatial configuration of ablation means carried thereon, or other working means which are to be spatially positioned. That therefore provides for orientation of the catheter equipped with reference units in that way, which is precise in three dimensions.

A further preferred embodiment of the invention is distinguished in that the reference units are coils or ultrasonic crystals mounted on or in the catheter. In that way the reference units can be embodied in a particularly simple fashion.

A further preferred embodiment of the invention is distinguished in that at least one reference unit is arranged at the catheter tip while the at least one further reference unit is arranged in the distal region of the catheter, wherein preferably a whole row of reference units, more preferably still between 12 and 24 reference units, are arranged in the distal region. The advantages of these embodiments with a plurality of reference units are that the increased number of reference units, in particular in the important distal region of the catheter, ensures an increased level of accuracy in terms of mapping - or if the catheter involves an ablation catheter—in terms of applying the lesion.

A further preferred embodiment of the invention is distinguished in that the distal region of the working catheter is of a previously established specific shape, preferably that of a circular arc, on which distal region at least three reference units are distributed, so that the specific, previously established shape of the distal region can be incorporated by the sensor device when ascertaining the position of the working catheter in positioning the working catheter. The advantages of this embodiment are in particular that just a small number of reference units, for example three reference units, and the use of a distal region involving a specific shape, for example a distal region which is in the shape of a circular arc, permit the position and orientation of the corresponding working catheter to be accurately determined.

A further preferred embodiment of the invention is distinguished in that the reference catheter may also involve a working catheter or the working catheter may also involve a reference catheter insofar as provided on each catheter are respective reference units for transmitting waves and respective reference units for receiving waves and/or respective reference units which can simultaneously transmit and receive the waves. The advantages of this embodiment are in particular that a system equipped with catheters which can be used flexibly in that way can also be used more flexibly. Thus, with this embodiment, if necessary it is possible to determine the position of the reference catheter by the working catheter acting as a reference catheter and vice-versa.

A further preferred embodiment of the invention is distinguished in that the sensor device, by means of the reference units according to the invention, can implement topological and/or electrical measurement of the endocardium in which the respective working catheter is disposed. The advantages of this embodiment are in particular that accordingly the precision of the working catheters according to the invention which are provided with a plurality of reference units can be used in succession or simultaneously in order to be able to precisely determine the anatomy of the endocardium and to be able to precisely electrically measure the corresponding heart cavity.

A further preferred embodiment of the invention is distinguished in that the sensor device ascertains the position of the working catheter in the coordinate system defined by the reference catheter by means of an electrical processing means, insofar as the sensor device, by means of the reference catheter, builds up at least an electromagnetic and/or an ultrasound field. The system according to the invention can be embodied in a particularly simple manner by means of an electromagnetic field.

A further preferred embodiment of the invention is distinguished in that the reference catheter can preferably be placed in the coronary sinus when using the system in the heart. This embodiment particularly advantageously guarantees that the reference catheter of the system according to the invention automatically compensates the movements of the patient and the heart of the patient. The implementation of the working operations by means of the working catheter, for example implementation of a mapping procedure or an ablation procedure, is thus effected in relation to a reference system which is disposed in the heart itself and which therefore moves therewith. An additional catheter for detecting the movement of the heart or of the patient is therefore no longer necessary.

A further preferred embodiment of the invention is distinguished in that the sensor device is so designed that, from the at least three reference units of the working catheter, it calculates a three-dimensional spline which represents the position of the working catheter in the co-ordinate system defined by the reference catheter. The advantages of this embodiment are that the spline which is calculated in that way can be superimposed on an image of the anatomy of the endocardium, which image was previously detected by means of a working catheter, so that the precise position of the working catheter in the heart cavity can be represented in a suitable display system, for example on the monitor, for the person operating the system.

A further preferred embodiment of the invention is distinguished in that the control device and/or the sensor device are provided in the respective catheters. The advantage of this embodiment is that in that way the respective catheters themselves already include in a fully integrated manner all necessary working means.

A further preferred embodiment of the invention is distinguished in that at least one of the reference units is in the form of a sensor for detecting the presence and/or the strength of the wall contact of an electrode of the working catheter with the endocardium surrounding the catheter. The advantages of this embodiment are that establishing the endocardial wall contact of the individual electrode means that it is possible to provide information as to whether the corresponding electrode of the working catheter is or is not bearing against the endocardium, so that it is possible to assess whether the corresponding reference units can be used for accurately detecting the anatomy of the endocardium. If the electrodes of the working catheter which at the same time can serve as reference units are bearing against the endocardium, then the anatomy of the endocardium is detected in that, in the region of the catheter which is in wall contact with the endocardium, the three-dimensional curve formed by the reference units which are in contact with the endocardium is used to generate a three-dimensional surface of the endocardium.

A further preferred embodiment of the invention is distinguished in that the system has at least two and preferably five working catheters, wherein each catheter has at least three and preferably between twelve and twenty four reference units which in a further preferred feature are in the form of electrodes, still more preferably in the form of ring electrodes, in order thus to detect the corresponding number of potential differences in the case of working catheters which are inserted into a heart cavity. The advantages of this embodiment are in particular that the anatomy and the associated excitation of the corresponding region of the heart can be detected in that way. It is also possible in that way to carry out investigations into the dynamics of the heart and transient phenomena in respect of excitation. The assembly of a plurality of catheters with a plurality of electrodes which are preferably in the form of pole rings and which serve as reference units, along a three-dimensional curve, forms almost a virtual cage catheter so that, in spite of using simple linear catheters, it is possible to attain the advantages of a cage catheter, in particular the possibility of a snapshot of cardiac excitation, as is necessary for example in particular in the case of tachycardia situations which occur for a brief period.

A further preferred embodiment of the invention is distinguished in that the working catheter is provided with a number of at least two electrodes which are preferably in the form of ring electrodes and which are mounted at different locations from the reference units on the working catheter, wherein in relation to the electrodes the reference units are in a previously established specific spatial position which can be taken into account by the sensor device when ascertaining the position of the working catheter in the co-ordinate system defined by the reference catheter. The advantages of this embodiment are in particular that the separation of reference units and electrodes makes it possible to exclude possible interactions between both electromagnetically operating components of the working catheters.

A further preferred embodiment of the invention is a system which includes a monitor serving as a display device for displaying the position, ascertained by the sensor device, of the working catheter in the co-ordinate system produced by the reference catheter. Such a monitor can represent both the three-dimensional structure of the endocardium surface ascertained by a mapping catheter, as a development, or as a three-dimensional object. Such a development or such a three-dimensional object can then be manipulated by the user, for example turned, so that the user can consider all sides. Such a 3-D structure therefore represents the anatomy of the corresponding endocardium. As already mentioned above however, the representation of the excitation, measured by a mapping catheter, of the corresponding cavity of the heart or the above-mentioned three-dimensional spline of the detected ablation catheter can also be projected on to a endocardium structure of that kind. The excitation can be represented for example in the form of an isochronous image calculated from potentials measured by the mapping catheter. Such an image represents the activation time on the endocardium surface. Then, by means of trigger algorithms, the beginning of the excitation at each electrode of the mapping catheter can be calculated from the measured potential variations. Those times which are calculated at individual locations of the endocardium are then interpolated for the remainder of the endocardium and represented so that for example a color corresponds to each activation time. An isochronous image can thus be obtained for each beat of the heart.

In another process, the so-called potential representation process, the measured potential is represented directly in color. In that case, a color is also associated with each potential value so that by virtue of a suitable choice of color it is possible to satisfactorily distinguish between the various states, for example ‘non-excited’, ‘excitation’, ‘begins’, and ‘excites’. In that case the values are interpolated between the electrodes in the isochronous representation. In that respect it has proven to be desirable for the representation of the potentials to be effected in an animation sequence in a slow motion camera as the excitation wave in real time passes very rapidly over the endocardium so that there is little point in time-synchronous representation as the user can only understand it with difficulty.

Further preferred embodiments of the invention are set forth in the appendant claims.

### BRIEF DESCRIPTION OF THE DRAWINGS

The invention will now be described in greater detail by means of embodiments with reference to the drawings in which:

FIG. 1 shows a system for determining the intracorporal position of a working catheter in accordance with a first embodiment,

FIG. 1a is a view in cross-section through the working catheter of FIG. 1,

FIG. 2 is a detailed illustration of a longitudinal slot of the distal end of a working catheter in accordance with a second embodiment, and

FIG. 2a is a view in cross-section through the working catheter of FIG. 2.

### DETAILED DESCRIPTION OF THE INVENTION

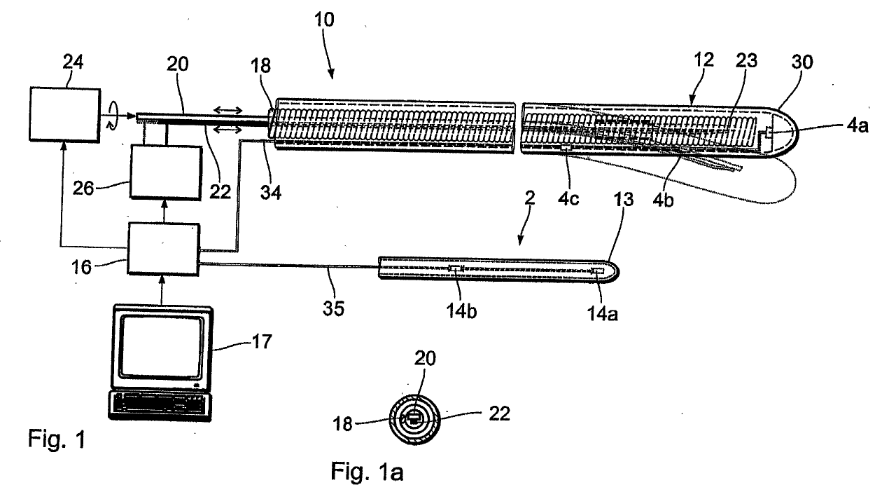

Referring to FIG. 1 shown therein is a system for determining the intracorporal position of a working catheter 10. In this case the system has a working catheter 10 and a reference catheter 2. Both the working catheter 10 and also the reference catheter 2 are adapted to be intracorporally introduced. The working catheter 10 in this case has three reference units 4a, 4b and 4c while the reference catheter 2 has two reference units 14a and 14b. The reference units 4a-4c and 14a, 14b are adapted to receive and/or transmit ultrasound waves or electromagnetic waves.

FIG. 1 shows in particular a working catheter 10 in accordance with a first embodiment whose distal end 12 can be laterally diverted by being deflected in any radial direction. That deflection is effected for example on the basis of the principle known from U.S. Pat. No. 5,254,088. For that purpose at its proximal end the working catheter 10 has two mechanical control drives or actuators 24 and 26 which are connected to a spiral sheath 18 which encloses a lumen and which is flexible at its distal end, and two control wires 20 and 22 which are guided in the lumen of the spiral sheath 18. The two mechanical control drives or actuators 24 and 26 are connected in known manner to the two control wires 20 and 22 in such a way that a rotation of the guide wires 20 and 22 with respect to the rest of the working catheter 10 and an axial movement of the control wires 20 and 22 relative to each other is possible, wherein the two control wires 20 and 22 are connected together at their distal end 23. Axial displacement of the control wires relative to each other by means of the actuator 26 produces lateral flexural deflection of the spiral sheath 18 and therewith the working catheter 10 in the flexible region of the spiral sheath 18 at the distal end 12 of the working catheter 10. Rotation of the control wires 20 and 22 is possible by means of the actuator 24.

The spiral sheath 18 is also arranged in the working catheter 10 rotatably relative thereto. A rotary movement of the spiral sheath 18 with the control wires 20 and 22 guided therein, with respect to the working catheter 10, can determine the radial direction of lateral deviation upon deflection of the distal end 12 of the catheter.

The working catheter 10 has three reference units 4a, 4b and 4c. In this case the first reference unit 4a is disposed at the distal tip 30 of the catheter while the second and third reference units 4a and 4b are in the distal region 12 of the catheter. Those reference units can be for example in the form of transducer units, in particular in the form of ultrasonic transducer units, ultrasonic crystals or piezoelectric crystals or coils and they are suitable for producing or receiving ultrasonic waves or electromagnetic waves. The three reference units 4a, 4b and 4c are connected to a control unit 16 by way of a signal line 34. It will be appreciated that it is also possible to provide more than two reference units 4b and 4c in the distal region 12 of the working catheter 10. For example between twelve and twenty four reference units can be provided in the distal region 12 of the working catheter 10.

Besides the working catheter 10 a reference catheter 2 is also shown in FIG. 1. In this case the reference catheter 2 has two reference units 14a and 14b in its distal region 13. In this case one reference unit 14a is arranged at the distal tip while a second reference unit 14b is arranged in the distal region 13 of the reference catheter 2. Those two reference units 14a and 14b are connected to the control unit 16 by way of a further signal line 35. The two reference units 14a and 14b, like the reference units 4a, 4b and 4c, are also suitable for producing and/or receiving ultrasonic waves or electromagnetic waves.

The reference units 4a-4c and 14a, 14b are arranged at previously established locations in the working catheter 10 and the reference catheter 2 respectively so that the relative position of the respective reference units 4a-4c and the respective reference units 14a and 14b relative to each other is known and can be taken into account when calculating position.

The control unit 16 receives signals from the reference units of the working catheter 10 and the reference catheter 2, in which respect either the reference units 14a, 14b of the reference catheter 2 emit ultrasonic waves or electromagnetic waves and the reference units 4a-4c of the working catheter 10 receive those waves, or the reference units 4a-4c of the working catheter 10 emit the waves and the reference units 14a, 14b of the reference catheter 2 receive the waves. By means of those received signals, the control unit 16 calculates the relative position of the working catheter 10 with respect to the reference catheter 2. On the basis of those ascertained position data, the control unit 16 further calculates a control signal for the actuators 24 and 26 for controlling the movement of the working catheter 20. Thus, by means of the control unit 16, it is possible to construct a closed or feed-back control system by which the working catheter 10 can be automatically moved to a desired position. In this respect it is particularly advantageous if the control unit 16 is designed to be programmable so that it is possible to input a desired intracorporal position to which the working catheter 10 is moved controlledly by means of the reference catheter 2. In particular the control unit 16 can calculate from the three reference units 4a-4c of the working catheter 10 a three-dimensional spline which represents the position of the catheter relative to the reference catheter.

In a simplified alternative configuration (not shown) the control unit is designed to display the position of the working catheter relative to the reference catheter and the working catheter is designed to be controllable by hand so that the physician can control the working catheter controllably by hand by means of the display.

The system according to the invention also has a computer together with a monitor 17 for displaying the three-dimensional structure of the endocardium surface, which is ascertained by means of the control unit 16, or the relative position of the working catheter 10.

FIG. 1a shows a view in cross-section through the working catheter 10 illustrated in FIG. 1. Shown herein are the spiral sheath 18 and the two control wires 20 and 22. In this case the control wire 20 is in the form of a flat band or strip whereby rotation of the catheter is simplified.

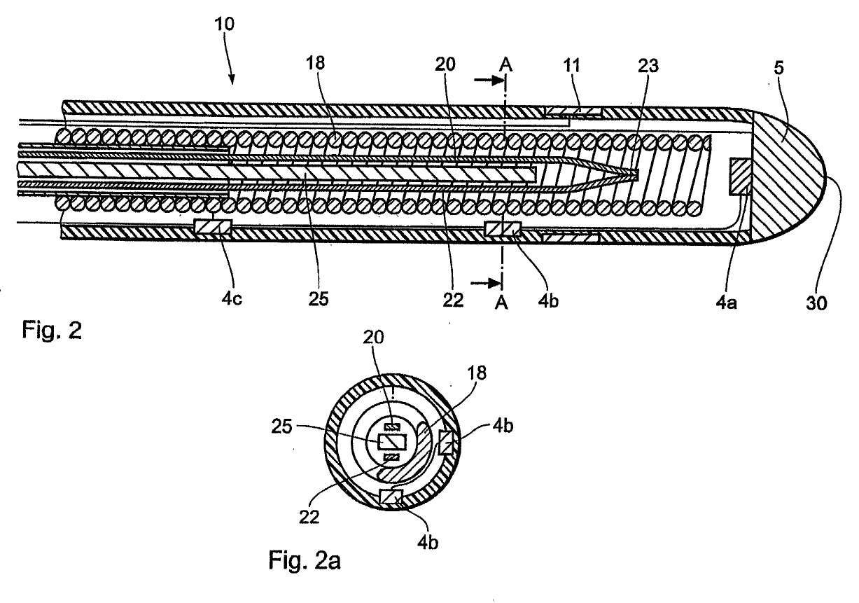

FIG. 2 shows the distal region 12 of a working catheter 10 in accordance with a second embodiment. In this case the structure of the distal end 12 of the working catheter 10 in accordance with the second embodiment corresponds to the structure of the distal end 12 of the working catheter 10 in accordance with the first embodiment in FIG. 1. In this case a tip electrode 5 is provided at the distal tip 30 of the working catheter 10. The catheter also has a ring electrode 11 in its distal region 12. In this case the tip electrode 5 and the ring electrode 11 can represent electrodes for mapping and/or for ablation of tissue. Preferably it is also possible for further electrodes, in particular ring electrodes, to be arranged in the distal region 12 of the working catheter 10. Those electrodes can be operated individually, jointly or in various combinations for mapping and/or for the ablation of tissue.

FIG. 2a is a view in cross-section taken along line AA in the distal region 12 of the working catheter 10 shown in FIG. 2. In this case the structure of the cross-section substantially corresponds to the structure of the cross-section in FIG. 1a. However a flat band or strip 25 is arranged between the control wires 20 and 22. The three reference units 4a, 4b of the catheter essentially form a triangle so as to permit exact three-dimensional positional determination.

What is claimed is:

1. A system for determining an intracorporal position of a working catheter, comprising
a working catheter for carrying out desired working operations, and
an intracorporal reference catheter for producing a co-ordinate system,
wherein the working catheter has a plurality of working catheter reference units for sending signals which are characteristic for the position of the working catheter, and
the reference catheter has a plurality of reference catheter reference units for receiving the signals sent by the working catheter reference units, and
a processing unit for calculating the position and an intracorporal orientation of the working catheter on the basis of signals received from the reference catheter reference units.

2. The system as set forth in claim 1
wherein the working catheter is a mapping catheter for generating a three-dimensional image of the heart cavity surrounding the mapping catheter.

3. The system of claim 1,
wherein the working catheter is an ablation catheter for producing a lesion of the endocardium surrounding the ablation catheter.

4. The system of claim 1,
wherein the working catheter is a catheter which can be fixedly implanted in a body and which carries electrodes of a cardiac pacemaker or a defibrillator.

5. The system of claim 1,
wherein the working catheter reference units are asymmetrically arranged on the working catheter so that the orientation of the working catheter can be detected in the co-ordinate system of the reference catheter.

6. The system of claim 1,
wherein the reference units are coils or ultrasonic crystals mounted on or in the catheter.

7. The system of claim 1,
wherein at least one reference unit is arranged at the catheter tip and
at least one further reference unit is arranged in the rest of the distal region of the catheter.

8. The system of claim 1,
wherein the distal region of the working catheter is of a previously established specific shape on which distal region at least three reference units are distributed so that the specific, previously established shape of the distal region can be incorporated by the processing unit when ascertaining the position of the working catheter by calculating the position of the working catheter.

9. The system of claim 1,
wherein either the reference catheter is also a working catheter or the working catheter is also a reference catheter, such that reference units for transmitting waves and reference units for receiving waves are provided on each catheter.

10. The system of claim 1,
wherein the processing unit is adapted by means of the reference units to implement topological and/or electrical measurement of the endocardium in which the respective working catheter is disposed.

11. The system of claim 1,
wherein the reference catheter reference units irradiate electromagnetic radiation and/or ultrasonic waves to ascertain the position of the working catheter in the co-ordinate system afforded by the reference catheter, wherein the reference catheter reference units build up at least one electromagnetic field.

12. The system of claim 1,
wherein the reference catheter is placed in the coronary sinus for use of the system in the heart.

13. The system of claim 1,
wherein the processing unit calculates a three-dimensional spline that represents the position of the working catheter in the reference catheter coordinate system from the data from the at least three working catheter reference units.

14. The system of claim 1,
wherein the processing unit is integrated in the respective catheters.

15. The system of claim 1,
wherein at least one of the reference units is a sensor for detecting the presence and/or the strength of the wall contact of the working catheter with the endocardium surrounding the catheter.

16. The system of claim 1,
wherein the system has between two and five working catheters, wherein each catheter has between three and twenty-four reference units which are electrodes to detect the corresponding number of potential differences in the case of working catheters inserted into a cavity in a heart.

17. The system of claim 16,
wherein the electrodes are ring electrodes.

18. The system of claim 1,
wherein the reference units are electrodes that are actuatable simultaneously by the processing unit.

19. The system of claim 1,
wherein the working catheter has at least two electrodes mounted on the working catheter at different locations from the reference units, wherein , relative to the electrodes, the reference units are in a previously established specific spatial position that can be taken into account by the processing units when ascertaining the position of the working catheter in the co-ordinate system defined by the reference catheter.

20. The system of claim 1, comprising
control members at the proximal end of the working catheter for producing a rotation of the working catheter and/or a flexing of the distal end of the working catheter.

21. The system of claim 20, comprising
a first signal line, extending from the distal tip to the proximal end of the working catheter and connecting to the working catheter reference units, and
a second signal line, extending from the distal tip to the proximal end of the reference catheter and connecting to the reference catheter reference units,
wherein the processing unit is connected by way of the first signal line to the working catheter reference units and by way of the second signal line to the reference catheter reference units, and
wherein the processing unit is connected to the control members actuates the control members in response to the signals from the reference catheter reference units in order to produce a rotation or a flexing of the working catheter.

22. A working catheter having a distal tip and a proximal end for use in a system as set forth in claim 1
characterised by
reference units for sending signals which are characteristic for the position of the working catheter, and
a signal line which extends from the distal tip to the proximal end of the working catheter and which is connected to the reference units.

23. A reference catheter having a distal tip and a proximal end for use in a system as set forth in claim 1
characterised by
reference units for receiving position signals, and
a signal line which extends from the distal tip to the proximal end of the reference catheter and which is connected to the reference units.

24. The system of claim 3,
wherein the ablation catheter produces a linear lesion.

25. The system of claim 2,
wherein the working catheter is a catheter which can be fixedly implanted in a body and which carries electrodes of a cardiac pacemaker or a defibrillator.

26. The system of claim 3,
wherein the working catheter is a catheter which can be fixedly implanted in a body and which carries electrodes of a cardiac pacemaker or a defibrillator.

27. The system of claim 4,
wherein the working catheter reference units are asymmetrically arranged on the working catheter so that the orientation of the working catheter can be detected in the co-ordinate system of the reference catheter.

28. The system of claim 25,
wherein the working catheter reference units are asymmetrically arranged on the working catheter so that the orientation of the working catheter can be detected in the co-ordinate system of the reference catheter.

29. The system of claim 26,
wherein the working catheter reference units are asymmetrically arranged on the working catheter so that the orientation of the working catheter can be detected in the co-ordinate system of the reference catheter.

30. The system of claim 5,
wherein the working catheter reference units are arranged to form the corners of a triangle.

31. The system of claim 27,
wherein the working catheter reference units are arranged to form the corners of a triangle.

32. The system of claim 28,
wherein the working catheter reference units are arranged to form the corners of a triangle.

33. The system of claim 29,
wherein the working catheter reference units are arranged to form the corners of a triangle.

34. The system of claim 5,
wherein the reference units are coils or ultrasonic crystals mounted on or in the catheter.

35. The system of claim 27,
wherein the reference units are coils or ultrasonic crystals mounted on or in the catheter.

36. The system of claim 28,
wherein the reference units are coils or ultrasonic crystals mounted on or in the catheter.

37. The system of claim 29,
wherein the reference units are coils or ultrasonic crystals mounted on or in the catheter.

38. The system of claim 6,
wherein at least one reference unit is arranged at the catheter tip and
at least one further reference unit is arranged in the rest of the distal region of the catheter.

39. The system of claim 34,
wherein at least one reference unit is arranged at the catheter tip and
at least one further reference unit is arranged in the rest of the distal region of the catheter.

40. The system of claim 35,
wherein at least one reference unit is arranged at the catheter tip and
at least one further reference unit is arranged in the rest of the distal region of the catheter.

41. The system of claim 36,
wherein at least one reference unit is arranged at the catheter tip and
at least one further reference unit is arranged in the rest of the distal region of the catheter.

42. The system of claim 37,
wherein at least one reference unit is arranged at the catheter tip and
at least one further reference unit is arranged in the rest of the distal region of the catheter.

43. The system of claim 7,
wherein the at least one further reference unit is a plurality of said reference units.

44. The system of claim 43,
wherein there are at least twelve said further reference units arranged in the rest of the distal region of the catheter.

45. The system of claim 43,
wherein there are fewer than twenty-four further reference units arranged in the rest of the distal region of the catheter.

46. The system of claim 8,
wherein the previously established specific shape is a circular arc.

47. The system of claim 9,
wherein either the reference catheter is also a working catheter or the working catheter is also a reference catheter , such that reference units for simultaneously transmitting waves and receiving waves are provided on each catheter.

48. The system of claim 9,
wherein the waves transmitted or received by the reference units are electromagnetic.

49. The system of claim 47,
wherein the waves transmitted or received by the reference units are electromagnetic.

50. The system of claim 9,
wherein the waves transmitted or received by the reference units are ultrasonic.

51. The system of claim 47,
wherein the waves transmitted or received by the reference units are ultrasonic.
